(19) United States
(12) Patent Application Publication
(10) Pub. No.: US 2005/0103350 A1
(43) Pub. Date: May 19, 2005

(54) Method of safely turning supine patient on to his or her side

(76) Inventor: Marian Henry, Kamloops (CA)

Correspondence Address: Steven Horowitz, Counselor At Law, Suite 700, 295 Madison Avenue, New York, NY, 10017, US

(21) Appl. No.: 10/696,171

(22) Filed: Oct. 28, 2003

(51) Int. Cl. A61B 19/00

(52) U.S. Cl. 128/869

(57) ABSTRACT

A method for turning a patient lying supine on a bed onto his side and avoiding musculoskeletal injuries to the care giver. First provide a draw sheet under the patient and a support sheet under the draw sheet, the support sheet on the bed, the draw sheet having a substantially frictionless lower surface and the support sheet having a substantially frictionless upper surface. Then a worker standing in a lunge position at a first side of the bed with palms facing upward grasps the first end of the draw sheet and pulls it horizontally to slide the patient on the bed toward the worker. Then the worker throws the first end of the draw sheet over the patient onto a second opposite side of the bed, walks to that side, and in lunge position with palms facing upward grasps the first end of the draw sheet and pulls it horizontally away from the patient.

### FIELD OF THE INVENTION

The field of this invention is methods of turning supine patients on to their side safely, and more particularly, methods that are safe to the health care or other worker who is doing the turning.

### BACKGROUND OF THE INVENTION AND DISCUSSION OF THE PRIOR ART

Patients lying in a hospital bed or in a bed in any personal care facility frequently needs to be turned to his or her side. This can be for the administration of medicine, a doctor's examination, skin breakdown, to prevent bed sores or even just plain comfort. Hundreds of thousands of these patients are physically unable to turn themselves and instead rely on health care workers. The frequency with which turning a patient on their bed occurs is such that it represents an occupational hazard for health care workers. Patients are heavy and if the worker turns the patient using a lifting motion without being properly situated, it can cause lower back injuries.

According to a June 2003 article in AAOHN Journal (vol. 51, no. 6), “[t]he high rate of musculoskeletal disorders experienced by workers in the health care industry has been and remains a major problem.” The same article notes that “[w]hen considering the event leading to an occupational injury in nursing and personal care facilities, overexertion specifically from lifting is a major contributing factor. In fact, the incidence rates calculated for overexertion as the cause for injuries in nursing and personal care facilities are four times higher than the national average for all industry. Back injuries among nurses and nurses' aides and others involved in direct client care are a major problem, which must be addressed.”

The article notes that the problem with back injuries is not merely the pain and medical result itself. Rather the cost for occupational injury is also a significant financial burden to the health care industry. Besides the cost for medical care and the compensation paid to the injured worker, there are indirect costs including replacement of the injured worker, additional training time by supervising and administration, loss of productivity and decreased morale.

Repetitive motions, such as pulling a draw sheet and lifting to turn a patient, could be the very motion contributing to the lower back injury. The care giver may be very good at performing this motion but that does not mean that such motion is good for the care giver. Very often, care givers are not aware of the trauma that their backs have sustained until irreparable damage has been done.

The prior art is full of methods and apparatuses that make it easier to move and turn patients lying on their side. For example, U.S. Pat. No. 1,334,901 to Higdon, U.S. Pat. No. 3,849,813 to Neoilson, U.S. Pat. No. 4,944,053 to Smith, U.S. Pat. No. 5,123,113 to Smith, U.S. Pat. No. 5,329,655 to Garner, U.S. Pat. No. 5,638,558 to Moore, U.S. Pat. No. 5,787,523 to Londberg and US 2001/0047543 A1 to Van Steenburg et al. relate to patient transfer devices and methods. The problem with the prior art is that they are not directed specifically to protect and assist the health care or other worker doing the dangerous lifting. Instead, they are designed to help the patient.

Accordingly, there is a compelling need for a patient turning method for turning patients onto their side that is specifically designed to eliminate musculoskeletal injuries in the health care workers doing the turning.

### SUMMARY OF THE PRESENT INVENTION

A method for turning a patient lying supine on a bed onto his side and avoiding musculoskeletal injuries. First provide a draw sheet under the patient and a support sheet under the draw sheet, the support sheet on the bed, the draw sheet having a substantially frictionless lower surface and the support sheet having a substantially frictionless upper surface. Then a worker standing in a lunge position at a first side of the bed with palms facing upward grasps the first end of the draw sheet and pulls it horizontally to slide the patient on the bed toward the worker. Then the worker throws the first end of the draw sheet over the patient onto a second opposite side of the bed, walks to that side, and in lunge position with palms facing upward grasps the first end of the draw sheet and pulls it horizontally toward him or herself.

### IMPORTANT OBJECTS AND ADVANTAGES

The following important objects and advantages of the present invention are:
(1) to provide a method of turning a patient lying on their back on a bed onto his or her side;
(2) to provide a method of turning someone onto their side in a manner that avoids musculoskeletal injuries to the worker or helper doing the turning;
(3) to provide a method of turning someone onto their side without any lifting motion;
(4) to provide a method of turning a patient lying supine on a bed using only horizontally applied force;
(5) to provide a safe method of turning someone on their side using two sheets;
(6) to provide a safe method of turning someone on their side that is easy to learn and easy to do;
(7) to provide a safe method of turning someone onto their side from a supine position that utilizes, in an alternative embodiment, a roll of fabric having two sheet portions;
(8) to provide a safe method turning someone lying supine on a bed onto his or her side;
(9) to provide a method of turning someone lying supine on a bed onto their side using two sheets that can tuck into the mattress;
(10) to provide a method of turning a supine individual onto his or her side that avoids lower back injuries for the health care worker doing the turning;
(11) to provide a method of turning a patient onto their side using an ergonomically correct technique;
(12) to provide a method of safely turning a supine patient while keeping the patient on the bed;
(13) to provide a safe method of turning a supine patient that does not require disposable objects;
(14) to provide a method for a health care worker working alone to turn a heavy patient from a supine position to their side;
(15) to provide a method of turning a patient onto their side that is easy enough to be performed by a non-professional;
(16) to provide a method of turning a patient that avoids having to handle the patient directly;
(17) to provide such a method that avoids accidentally bruising the patient;
(18) to provide a method of turning a patient that is easily affordable;
(19) to provide a method of repositioning a patient in a bed in any direction; and
(20) to provide a method of repositioning a patient that prevents bedsores and skin breakdown.

### BRIEF DESCRIPTION OF THE DRAWINGS

FIG. 1 is a top plan view of a patient lying supine in a bed in the first step of the method of the present invention;

FIG. 2 is an end view of FIG. 1;

FIG. 3 is a top plan view showing a health care worker performing the first pulling step of the method of the present invention;

FIG. 4 is an end view of FIG. 3;

FIG. 5 is a top plan view showing a health care worker throwing the draw sheet over the patient in accordance with the method of the present invention;

FIG. 6 is an end view of FIG. 5;

FIG. 7 is a top plan view showing the health care worker in position to perform the final pulling motion of the method of the present invention;

FIG. 8 is an end view of FIG. 7 after the pulling motion;

FIG. 9 is an end view of a roll of fabric apparatus used in an alternative method of the present invention;

FIG. 10 shows a top plan view of an alternative embodiment of the draw sheet used in the method of the present invention.

### DETAILED DESCRIPTION OF THE PREFERRED EMBODIMENT

The method of the present invention will now be illustrated by reference to the accompanying drawings.

“Lunge position” is defined to mean one foot, the left or the right, forward from a normal standing position and the other foot backward from a normal standing position. This is best illustrated by the stick figures in FIGS. 4, 6 and 8. The term “normal friction” referring to a surface, material or object simply means the surface, material or object is not a special material that has low friction or is substantially frictionless. A “substantially frictionless” surface, material or object simply means a low enough friction characteristic when dragged adjacent a similar material sufficient to carry out the steps of the method being described. For example, when pulling a weighted (a patient is resting thereon) smooth nylon sheet resting on another similar smooth nylon sheet, there is almost no friction, or “low” friction comparatively and the sheet moves quickly once the force is exerted. The preferred low friction or substantially frictionless material to be used in the method of the present invention is lightweight nylon. Specifically, it has been found that a particular lightweight nylon called 86 pic nylon taffeta performs superbly in terms of low friction effect when moved against an identical piece of such nylon pursuant to the method of the present invention. Pursuant to allowable industry accepted deviation, 86 pic nylon taffeta, has a thread count of between 84 and 88 pic. However, the method of the present invention certainly contemplates using other nylon material in central portions 14, 24 of draw sheet 10 and support sheet 20, and indeed other low friction materials.

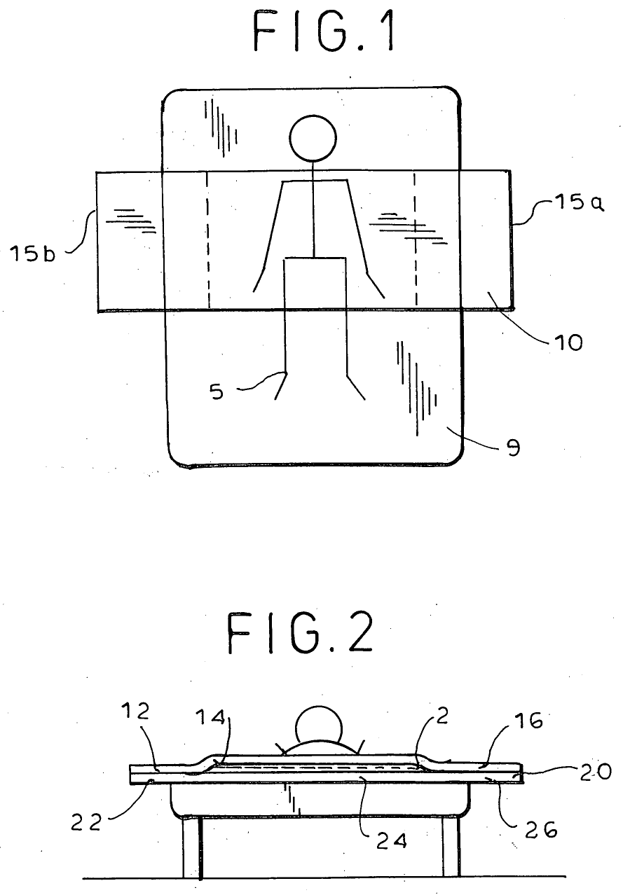

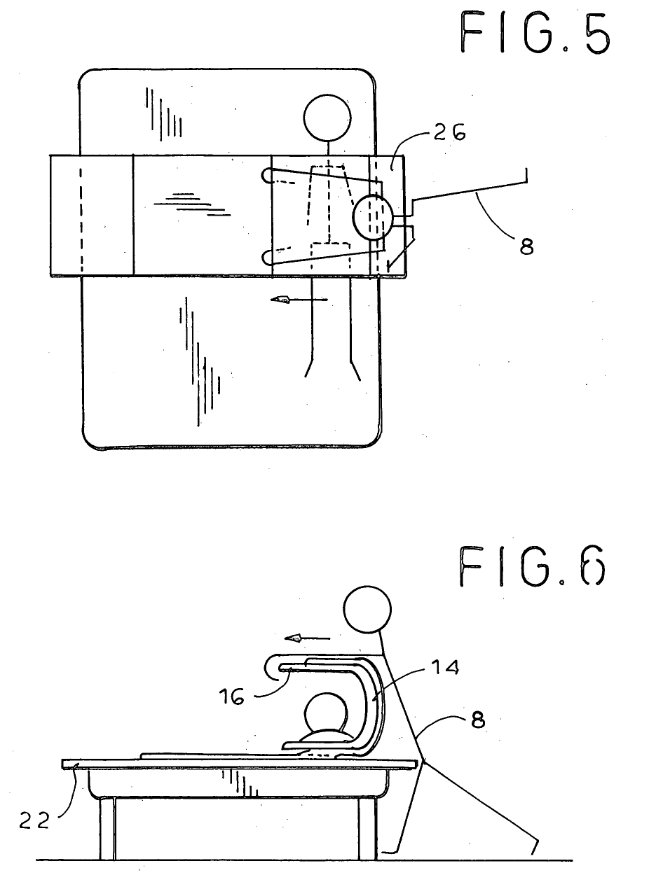

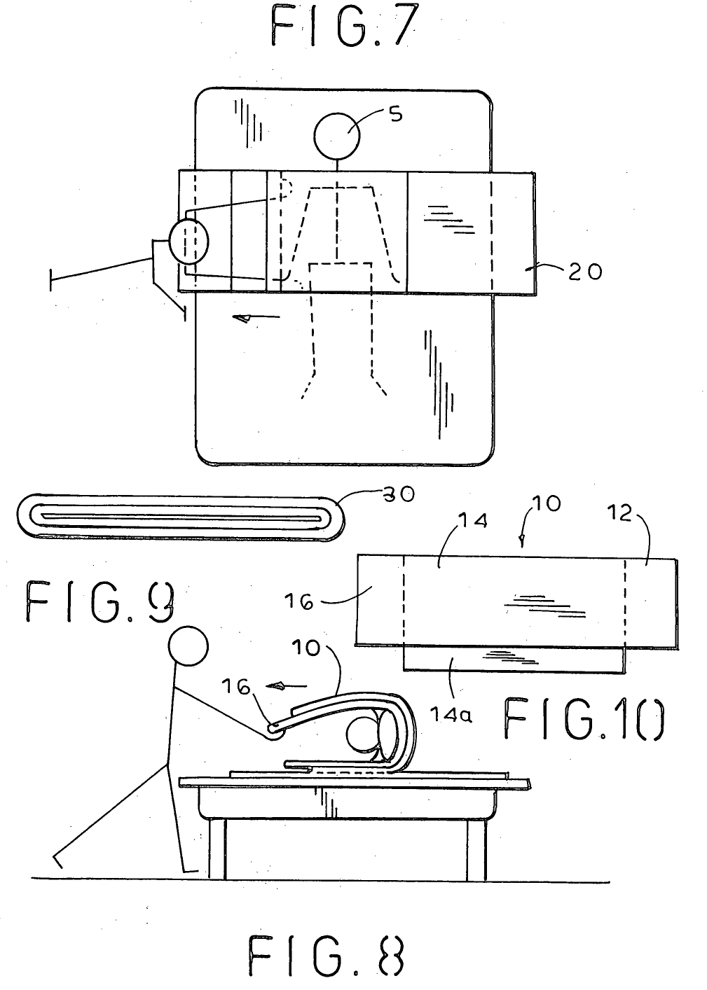

As seen from FIGS. 1-10, a patient is lying on a bed on his or her back, which is called “supine position”. Although not shown in any of the drawing figures, there is typically one or more layers of bed sheets between the patient and the bed. In the first step of the method of the present invention, step (a) in a preferred embodiment, one provides two separate sheets under the patient and above the bedding that is on the bed. A draw sheet 10 is provided directly under the patient and a support sheet 20 is provided directly under the draw sheet 10. Support sheet 20 is positioned directly on top of any bedding on the bed.

Draw sheet 10 has a left portion 12, a central portion 14 and a right portion 16. Left and right portions 12, 16 of draw sheet 10 are normal friction materials, such as cotton or whatever other materials bed sheets or bedding in a hospital or personal care facility are made of. Central portion 14 of draw sheet 10 is substantially frictionless with respect to its lower surface. That is accomplished by taking a normal friction draw sheet such as a cotton sheet and sewing onto a central portion thereof on side of said central portion a low friction (“substantially frictionless”) material 2 such as smooth nylon. That side of the central portion will then be used as the substantially frictionless lower surface of the central portion of the draw sheet in the steps of the method of the present invention.

The left and right portions 12, 16 of draw sheet 10 are provided within draw sheet 10 to provide an easy point from which to grasp draw sheet 10. Generally, it is easier to grasp a normal sheet than a smooth nylon sheet or a sheet made of another substantially frictionless material. Furthermore, while theoretically central portion 14 could be one layer of substantially frictionless material and the method of the present invention could be effectuated, that would create an incidental deleterious effect in that the patient would be lying on smooth nylon or another non-breathable material. Accordingly, that is definitely a disadvantageous option.

The central portions 14, 24 of draw sheet 10 and support sheet 20 need only be long enough to cover the width of the patient plus at least a few more inches on each side to take into consideration shifting or movement of the patient left or rightward on the bed prior to use of the method or during the method. In a preferred embodiment, central portions 14, 24 should be significantly longer than the width of the patient to take into consideration the above-mentioned shifting.

Support sheet 20 has a substantially frictionless upper surface that is designed to be held alongside the substantially frictionless lower surface of central portion 14 of draw sheet 10. In order to accomplish this in the simplest manner, support sheet 20 is made the same way draw sheet 10 is made—by taking a normal friction sheet and attaching to one side/surface (what will become the upper surface of the support sheet 20) of a central portion thereof a low friction material. Thus support sheet 20 has central portion 24 and left and right portions 22, 26. It is noted that the dimensions of draw sheet 10 and support sheet 20 are preferably the same and the dimensions of their respective central portions are preferably the same—that simplifies ensuring that the two low friction surfaces are adjacent to one another during the first step of the method of the present invention.

Draw sheet 10 will come in multiple sizes since the length from one patient's hips to their shoulders varies from patient to patient. Draw sheet is thus sized for the patient so that a width of draw sheet 10 approximately spans at least an approximate distance from the patient's neck to just past the patient's hips, and in a preferred embodiment it spans an approximate distance from the patient's neck to just past the patient's hips. Draw sheet 10 is long enough that a first end of the draw sheet 10 which is also at the end of right portion 16 extends beyond the edge of the bed. This makes grasping draw sheet 10 easy and avoids having the worker have to bend over into an ergonomically dangerous position for the back when grasping draw sheet 10. Furthermore, draw sheet 10 and support sheet 20 are long enough that both the right and left portions of each 12, 16, 22, 26 can be tucked into the bed when not in use.

Prior to beginning step (b) of the method of the present invention, the care giver makes sure that side rail on the unattended side of the bed (the side that no care giver is standing on during steps (b) and (e)) is in an “UP” position and that the bed is raised to the worker's waist height. Accordingly, notwithstanding the appearance of the height of the bed in the drawing figures, the bed is in actually raised to waist height. The rail cannot be “UP” on the side where the care giver is working.

As seen in FIG. 10, as an option, central portion of draw sheet 10 can along its width also have an extension 14a on one side to avoid the patient's feet contacting the nylon material.

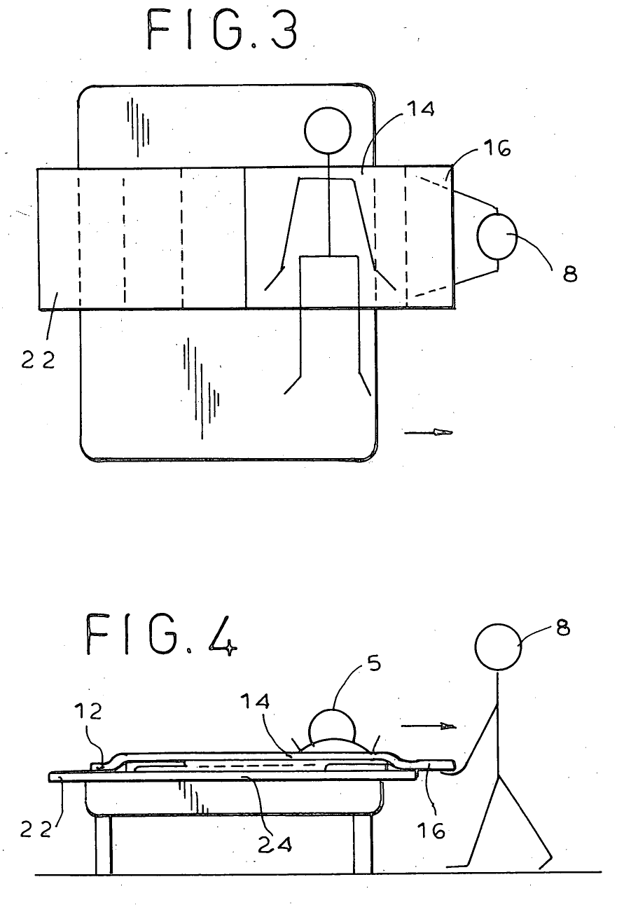

Once draw sheet 10 and support sheet 20 are properly in position, in step (b) of the method, the care giver (sometimes called the “worker” for convenience) stands in a lunge position at a first side of the bed with palms facing upward. Palms upward is safest for the care giver though the natural instinct is to grab something with the palms down. The care giver grasps the first end of the draw sheet and pulls it in a horizontal motion away from the patient and toward himself. As a result, the patient slides toward the first side of the bed. This is illustrated in FIGS. 3-4. The term “in a horizontal motion” in this patent application means that the draw sheet is pulled along a direction approximately parallel to the ground or the surface of the bed.

In the next step, step (c) of the method of the present invention, the care giver 8 throws the first end of the draw sheet 10 over the patient 5 onto a second opposite side of the bed 9. It should be understood clearly that the term “throwing” is intended to encompass any manner of causing the draw sheet to be repositioned so that the first end of said draw sheet 10 is located on the second opposite side of the bed. In a preferred embodiment, this is done by simply throwing the first end of the draw sheet 10. Conceivably, however, other means can be employed such as using some utensil to carry said first end of the draw sheet 10 to the opposite side of the bed. In step (d) of the method of the present invention, the worker then walks to the second opposite side of the bed. Again, any means by which the care giver ends up on the opposite side of the bed is contemplated by the present invention. Walking is simply the simplest and preferred method.

In the last step of the method of the present invention, step (e) the care giver or worker stands in a lunge position at the second opposite side of the bed with palms facing upward and grasps the first end of the draw sheet and pulls the first end of the draw sheet horizontally away from the patient, meaning toward him or herself. As a result, the patient turns on his or side.

It is emphasized that the care giver engaging in steps (b) and (e) of the method, the steps involved in actually pulling the draw sheet 10 and making the patient move, should be pulling gently and only in a horizontal direction. There should be no lifting or pulling upward. This is exactly the motion the care giver should avoid to prevent a musculoskeletal injury.

In an alternative embodiment of the steps of the method of the present invention, in step (a) of the method provide a flattened roll of fabric under the patient, the flattened roll of fabric having an inner layer and an outer layer, the inner layer made of a substantially frictionless material and the outer layer made of a normal friction material. Draw sheet portion of the outer layer of the roll of fabric is the portion of the outer layer that is directly under the patient. Once again, the flattened roll of fabric is directly on top of a bed and any bedding of the bed. The flattened roll of fabric is sized for the patient so that a width of the flattened roll of fabric approximately spans at least an approximate distance from the patient's neck to just past the patient's hips. A first end of the draw sheet portion of the flattened roll of fabric extends beyond the bed for comfortable grasping. Roll 30 of fabric is shown in FIG. 9. In this embodiment the nylon forms a continuous inner layer spanning the entire length of the roll. Then the method is carried out essentially in the same manner as the preferred embodiment of the method.

For example, in step (b) of the method, the care giver or worker stands in a lunge position at a first side of the bed with palms facing upward grasping the first end of the draw sheet portion of the flattened roll of fabric and pulls the first end of the draw sheet portion in a horizontal motion toward him or herself, with the patient sliding as a result toward the first side of the bed. Steps (c), (d) and (e) continue as before. In step (c) the worker throws the first end of the draw sheet portion over the patient onto a second opposite side of the bed, in step (d) the worker walks to the second opposite side of the bed, and in step (e) the worker stands in a lunge position at the second opposite side of the bed with palms facing upward grasping the first end of the draw sheet portion and pulling the first end of the draw sheet portion in a horizontal motion toward him or herself so that the patient turns on his or her side as a result.

In effect, the care giver grasps a top layer of the roll of fabric at the point where the first end of the draw sheet is located and the care giver pulls in the same manner toward him or herself. In this case, step (b) must involve pulling a sufficient length of the roll to allow step (c) to occur properly. The length of the flattened roll of fabric (which is the same as the length of the draw sheet portion of the flattened roll of fabric) must sufficiently exceed the distance that the patient slides in step (b) that enough slack is produced to throw over the patient in step (c) of the method. It is estimated that therefore to produce the required slack in roll 30 the length of draw sheet portion 10 (and of support sheet portion 20) should be substantially more than twice the anticipated distance that the patient will move in step (b) of the method.

It should be noted that the above method is compatible with other methods used by care givers. For example, for incontinence concerns, whatever method is currently used to protect the bed sheets is used and placed over the top surface of the draw sheet 10.

In general, it is helpful to explain the method to the patient prior to doing it.

Repositioning Patients

In addition to turning patient to the side from a supine position, the method of the present invention can also be adapted to move a supine patient from one portion of the bed to another either laterally or vertically. If movement is side to side, just perform steps (a) and (b) of the method previously described.

Often, a patient slides down in their bed and needs to be repositioned upward in the bed. Alternatively, the patient is too far upward in their bed and needs to be repositioned downward. If movement is to be up or down the bed, then two workers are needed. In step (a) of the repositioning method, provide the draw sheet 10 and support sheet 20 as in step (a) of the method of turning the supine patient on to his or her side.

Step (b) is then performed as before but instead of a single worker or care giver on a first side of the bed in a lunge position palms facing up, there is a care giver on the first and second opposite sides of the bed in a lunge position with palms facing up. The first worker at a first side of the bed grasps the first side 15a of draw sheet 10 near the first end of the draw sheet with palms facing up and standing so that he or she is facing the first side of the draw sheet (the first worker's feet face in a direction of the length of the bed), the second worker at the second side of the bed grasps the second side 15b of draw sheet 10 near the second end of the draw sheet with palms facing up and standing facing the second side of the draw sheet (the second worker's feet face in a direction of the length of the bed). The workers, synchronize their behavior (such as by counting 1-2-3) so that they then simultaneously pull the draw sheet up or down the bed to reposition the patient, the patient sliding as a result up or down the bed. The workers' pulling motion is thus always toward themselves, and that avoids overextending themselves. Alternatively, with respect to the method for vertical repositioning, instead of a lunge position, the care givers can have one foot flat on the floor and the second foot lifted with its knee up on the bed. In this case too the workers' pulling motion is always toward themselves to avoid overextending themselves.

In the case of vertical repositioning a pillow is placed at the head of the bed to prevent bumping patient's head prior to step (b) being performed.

It is to be understood that while the apparatus of this invention have been described and illustrated in detail, the above-described embodiments are simply illustrative of the principles of the invention. It is to be understood also that various other modifications and changes may be devised by those skilled in the art which will embody the principles of the invention and fall within the spirit and scope thereof. It is not desired to limit the invention to the exact construction and operation shown and described. The spirit and scope of this invention are limited only by the spirit and scope of the following claims.

1. A method for turning a patient lying supine on a bed onto his or her side in a manner that avoids musculoskeletal injuries to workers, the method comprising the steps of:
(a) providing a draw sheet under the patient and providing a support sheet directly under the draw sheet, the support sheet positioned directly on top of a bed and any bedding of the bed, a central portion of the draw sheet having a substantially frictionless lower surface and a normal friction upper surface, and a normal friction surface in a left portion and in a right portion of the draw sheet and the support sheet having a substantially frictionless upper surface in a central portion, a length of the central portion of the draw sheet and a length of the central portion of the support sheet exceeding a width of the patient, the draw sheet sized for the patient so that a width of the draw sheet approximately spans at least an approximate distance from the patient's neck to just past the patient's hips, the draw sheet long enough so that a first end and a second end thereof can be tucked into the bed and a first end easily grasped,
(b) a worker standing in a lunge position at a first side of the bed with palms facing upward grasping the first end of the draw sheet and pulling said first end of the draw sheet in a horizontal motion toward him or herself, the patient sliding as a result toward the first side of the bed,
(c) the worker throwing the first end of the draw sheet over the patient onto a second opposite side of the bed,
(d) the worker walking to the second opposite side of the bed,
(e) the worker standing in a lunge position at the second opposite side of the bed with palms facing upward grasping the first end of the draw sheet and pulling said first end of the draw sheet in a horizontal motion toward him or herself, the patient turning on his or her side as a result.

2. The method of claim 1, wherein the pulling in steps (b) and (e) are done gently.

3. The method of claim 1, wherein step (a) involves providing a draw sheet in which the substantially frictionless material of the central portion of the draw sheet is nylon

4. The method of claim 1, wherein step (a) involves providing a draw sheet in which the substantially frictionless material of the central portion of the draw sheet is nylon and wherein the pulling in steps (b) and (e) are done gently.

5. A method for turning a patient lying supine on a bed onto his or her side in a manner that avoids musculoskeletal injuries to workers, the method comprising the steps of:
(a) providing a flattened roll of fabric under the patient, the flattened roll of fabric having an inner layer and an outer layer, a draw sheet portion of the outer layer of the flattened roll of fabric directly under the patient and the flattened roll of fabric directly on top of a bed and any bedding of the bed, the inner layer made of a substantially frictionless material and the outer layer made of a normal friction material surface, the flattened roll of fabric sized for the patient so that a width of the flattened roll of fabric approximately spans at least an approximate distance from the patient's neck to just past the patient's hips, a first end of the draw sheet portion of the flattened roll of fabric extending beyond the bed for comfortable grasping,
(b) a worker standing in a lunge position at a first side of the bed with palms facing upward grasping the first end of the draw sheet portion of the flattened roll of fabric and pulling said first end of the draw sheet portion in a horizontal motion toward him or herself, the patient sliding as a result toward the first side of the bed,
(c) the worker throwing the first end of the draw sheet portion over the patient onto a second opposite side of the bed,
(d) the worker walking to the second opposite side of the bed,
(e) the worker standing in a lunge position at the second opposite side of the bed with palms facing upward grasping the first end of the draw sheet portion and pulling said first end of the draw sheet portion in a horizontal motion toward him or herself, the patient turning on his or her side as a result.

6. The method of claim 5, wherein the pulling in steps (b) and (e) are done gently.

7. The method of claim 5, wherein step (a) involves providing a draw sheet in which the substantially frictionless material of the central portion of the draw sheet is nylon

8. The method of claim 5, wherein step (a) involves providing a draw sheet in which the substantially frictionless material of the central portion of the draw sheet is nylon and wherein the pulling in steps (b) and (e) are done gently.

9. A method for repositioning a patient lying supine on a bed in a manner that avoids musculoskeletal injuries to workers, the method comprising the steps of:
(a) providing a draw sheet under the patient and providing a support sheet directly under the draw sheet, the support sheet positioned directly on top of a bed and any bedding of the bed, a central portion of the draw sheet having a substantially frictionless lower surface and a normal friction upper surface, and a normal friction surface in a left portion and in a right portion of the draw sheet and the support sheet having a substantially frictionless upper surface in a central portion, a length of the central portion of the draw sheet and a length of the central portion of the support sheet exceeding a width of the patient, the draw sheet sized for the patient so that a width of the draw sheet approximately spans at least an approximate distance from the patient's neck to just past the patient's hips, the draw sheet long enough so that a first end and a second end thereof can be tucked into the bed and a first end easily grasped, and
(b) a first worker standing in a lunge position at a first side of the bed with palms facing upward and standing facing a first side of the draw sheet grasping the first side of the draw sheet near the first end of the draw sheet and pulling the draw sheet at the same time that a second worker standing in a lunge position at a second side of the bed with palms facing upward and standing facing a second side of the draw sheet near the second end of the draw sheet grasping the second side of the draw sheet is pulling the draw sheet, both workers pulling the draw sheet toward themselves so that the patient slides upward or downward in the bed.

10. The method of claim 9, wherein the pulling in step (b) is done gently.

11. The method of claim 9, wherein step (a) involves providing a draw sheet in which the substantially frictionless material of the central portion of the draw sheet is nylon

12. The method of claim 9, wherein step (a) involves providing a draw sheet in which the substantially frictionless material of the central portion of the draw sheet is nylon and wherein the pulling in step (b) is done gently.

13. A method for repositioning a patient lying supine on a bed in a manner that avoids musculoskeletal injuries to workers, the method comprising the steps of:
(a) providing a draw sheet under the patient and providing a support sheet directly under the draw sheet, the support sheet positioned directly on top of a bed and any bedding of the bed, a central portion of the draw sheet having a substantially frictionless lower surface and a normal friction upper surface, and a normal friction surface in a left portion and in a right portion of the draw sheet and the support sheet having a substantially frictionless upper surface in a central portion, a length of the central portion of the draw sheet and a length of the central portion of the support sheet exceeding a width of the patient, the draw sheet sized for the patient so that a width of the draw sheet approximately spans at least an approximate distance from the patient's neck to just past the patient's hips, the draw sheet long enough so that a first end and a second end thereof can be tucked into the bed and a first end easily grasped, and
(b) a worker standing in a lunge position at a first side of the bed with palms facing upward grasping the first end of the draw sheet and pulling said first end of the draw sheet in a horizontal motion toward him or herself, the patient sliding as a result toward the first side of the bed.

14. The method of claim 13, wherein the pulling in step (b) is done gently.

15. The method of claim 13, wherein step (a) involves providing a draw sheet in which the substantially frictionless material of the central portion of the draw sheet is nylon

16. The method of claim 13, wherein step (a) involves providing a draw sheet in which the substantially frictionless material of the central portion of the draw sheet is nylon and wherein the pulling in step (b) is done gently.
